(19) United States
(12) Patent Application Publication
(10) Pub. No.: US 2024/0408159 A1
(43) Pub. Date: Dec. 12, 2024

(54) PHYTOENE-CONTAINING OLEORESIN AND COMPOSITION

(71) Applicant: LYCORED LTD., Be'er Sheva (IL)

(72) Inventors: Tal OFFER, Giv'at Ada (IL); Etgar LEVY-NISSENBAUM, Ramat-HaSharon (IL); Masha SAPOJNIK, Be'er Sheva (IL); Oleg BRAVERMAN, Be'er Sheva (IL); Iryna BONDAR, Verkhnedneprovsky District (UA)

(21) Appl. No.: 18/701,781

(22) PCT Filed: Oct. 16, 2022
(86) PCT No.: PCT/IL2022/051092

§ 371 Date: Apr. 16, 2024

Related U.S. Application Data
(60) Provisional application No. 63/256,588, filed on Oct. 17, 2021.

(51) Int. Cl. A61K 36/06; A61K 8/31; A61K 8/63; A61K 8/92; A61K 8/9728; A61K 9/00; A61K 9/48; A61K 31/01; A61K 31/575; A61K 36/185; A61P 17/16; A61Q 17/04; A61Q 19/08

(52) U.S. Cl. CPC A61K 36/06; A61K 8/31; A61K 8/63; A61K 8/922; A61K 8/9728; A61K 9/0014; A61K 9/48; A61K 31/01; A61K 31/575; A61K 36/185; A61P 17/16; A61Q 17/04; A61Q 19/08

(57) ABSTRACT

The present invention provides a solvent-free, non-GMO oleoresin obtained from Blakeslea trispora, comprising phytoene at a concentration of 10-40% (w/w). In addition, the invention is directed to compositions and to topical and oral dosage forms containing said oleoresin. The use of said compositions and dosage forms in the treatment and prevention of various skin conditions and for improving skin quality is also disclosed and claimed.

### FIELD OF THE INVENTION

The present invention is related to an oleoresin comprising a high concentration of phytoene, and to a composition comprising said phytoene together with jojoba oil. The invention is also directed to the use of said phytoene-containing composition in the prevention and management of skin lesions caused by exposure to air pollution or UV radiation.

### BACKGROUND OF THE INVENTION

Phytoene is a largely colourless carotenoid that is found in many different species of fruit. As well as acting as a key intermediate in the biosynthesis of other bioactive carotenoids, phytoene itself possesses several biological properties, including anti-inflammatory, antioxidant, and UV-absorbing activities. These properties, combined with its lack of color, have led to the development of topical and cosmetic preparations comprising phytoene for use in the treatment or prevention of various skin conditions. One example of such a preparation that may be used for lightening or whitening skin color is described in U.S. Pat. No. 8,398,958.

Phytoene may be obtained from a number of different sources including various fruit species (e.g., the tomato), algae (e.g., Dunaliella species) and moulds, the most notable of which is Blakeslea trispora.

B. trispora is a pathogen of tropical plants, but harmless to animals and humans. This species has been used extensively to study the carotenoid synthetic pathway, since it has been found to produce important carotenoids such as lycopene, beta-carotene, phytoene and phytofluene. In addition, it has been found that it is possible, by means of strain selection and growth conditions to produce high quantities of phytoene from B. trispora, as described in Ukrainian patent UA85489. Furthermore, UA86556 discloses a method for obtaining a phytoene-containing biomass following incubation of the strain described in UA85489.

In recent years, the role of airborne pollution in causing disease has become a matter of increasing concern. While the impact of airborne pollutants on cardiac and respiratory health have been known for a long time, it is now becoming clear that the skin is often targeted and adversely affected by such pollutants. Thus, it has been found that is a clear correlation between increases in airborne pollution and the number of new cases of acne, atopic dermatitis, urticaria and other skin conditions in humans. In addition, airborne pollution is also considered to be an exacerbating factor in accelerated or premature skin ageing. Finally, there is an increasing body of knowledge linking air pollution to the incidence and/or severity of various skin cancers.

While airborne pollution may come from many different sources, the pollutants contained in motor vehicle exhaust are particularly significant, in view of the daily exposure of a very large percentage of the world population to such pollution. Of particular concern are the pollutants contained in the exhaust emitted by diesel engines.

Diesel exhaust contains a very large number of compounds, many of which are associated with significant health risk. In addition to the negative effects caused by compounds that are dissolved in the gaseous phase, diesel exhaust also contains a significant concentration of diesel particulate matter (DPM). These particles, which exist in various different size ranges, are of particular concern with regard to lung cancer and other serious pulmonary diseases. However, recent studies have also shown a direct connection between exposure of skin cells to DPM and various skin conditions (such as those mentioned above). In addition, several studies describing the effects of airborne particulate matter (including DPM) on skin cells have been published. One such example is the study by Ryu et al. [Experimental & Molecular Medicine 51:108; 2019], in which the authors describe the biochemical changes underlying keratinocyte senescence caused by exposure to airborne particulate matter. Other studies have also described the increase in levels of cytokines and interleukins, including IL-8, when cultured cells are exposed to DPM. Thus, for example, Reynolds et al. (2011) [Environmental Health Perspective 119 (3): 332-336] describe the biochemical pathways underlying the increase in IL-8 secretion that is seen when epithelial cells are exposed to DPM.

A need exists for a safe, naturally derived composition that may be used to prevent, alleviate, or treat skin conditions caused by, or related to, exposure to airborne pollutants, particularly those found in diesel exhaust, as well skin conditions related to sunlight exposure.

### SUMMARY OF THE PRESENT INVENTION

In one aspect of the present invention, the present inventors have discovered that it is possible to manufacture a solvent-free, non-GMO oleoresin obtained from the mold species Blakeslea trispora, comprising phytoene at a concentration far higher (e.g., 20-30-fold higher) than previously obtained. Furthermore, it was unexpectedly found that there is a selective enrichment of the phytosterol ergosterol in the presently disclosed oleoresin, when compared with the biomass from which said composition is obtained. This phytoene-enriched oleoresin is used, in certain embodiments as the starting material for the preparation of the compositions and dosage forms disclosed and described in more detail, hereinbelow.

In a further aspect, the inventors have found that a composition comprising phytoene in combination with Simmondsia Chinensis (Jojoba) Seed Oil is particularly advantageous for use in the protecting the skin from damage caused by airborne pollutants (such as diesel particulate matter (DPM)) and UV radiation, and for maintaining and improving skin quality. The present invention is, therefore, primarily directed to a solvent-free, non-GMO oleoresin obtained from Blakeslea trispora, comprising phytoene at a concentration of 10-40% (w/w).

In one preferred embodiment, the concentration of phytoene in the oleoresin is in the range of 20-40% (w/w).

In another preferred embodiment, the concentration of phytoene in the oleoresin is in the range of 20-30% (w/w).

In many cases, the above-disclosed oleoresin will comprise one or more phytosterols. One of the major phytosterols present in the oleoresin is ergosterol, which is generally present at a concentration in the range of about 0.5 to 1% (w/w). In one preferred embodiment, the ergosterol concentration is about 0.6% (w/w).

It is to be noted that the term “about” is used throughout this disclosure (in relation to percentage concentration values) to indicate that concentrations that differ from the stated values by up to +/−0.5% are included within the scope defined by the stated concentration value or range.

In another aspect, the present invention is directed to a solvent-free, non-GMO oleoresin obtained from Blakeslea trispora, comprising phytoene at a concentration of 20-30% (w/w), obtained or obtainable by means of a process comprising the steps of:
a) Cultivating the mold species Blakeslea trispora;
b) Obtaining a biomass from said cultivated B. trispora;
c) Subjecting said biomass to supercritical carbon dioxide fluid extraction performed at an operating pressure of between 300 and 450Bar, and an operating temperature of about 50° C.-65° C., thereby yielding phytoene-containing oleoresin.

Preferably, the Blakeslea trispora mold that is cultivated in step (a) of the above-disclosed method is a phyt 1+/phyt 1−strain.

Generally, the oleoresin obtained by this process comprises phytoene at a concentration of 20 to 30% (w/w) and one or more phytosterols. In most embodiments, the one or more phytosterols comprises ergosterol as a major component. The concentration of ergosterol in the oleoresin is generally in the range of 0.5 to 1% (w/w). In one preferred embodiment, the ergosterol concentration in the oleoresin is about 0.6% (w/w).

In a further aspect, the present invention is directed to a composition comprising a phytoene-containing oleoresin as disclosed hereinabove, together with seed oil obtained from Simmondsia chinensis (Jojoba). In one preferred embodiment, the concentration of phytoene in said composition is in the range of 0.01 to 10% (w/w). In a particularly preferred embodiment, the phytoene concentration is in the range of 1 to 2% (w/w), more preferably in the range of 1 to 1.5% (w/w). In some preferred embodiments, the concentration of phytoene in the composition is about 1% (w/w).

In a still further aspect, the present invention is directed to a dosage form comprising the above-disclosed phytoene-containing composition, together with one or more pharmaceutically-acceptable, nutraceutically-acceptable or cosmetically-acceptable excipients.

In another aspect, the present invention is directed to a dosage form comprising the above-disclosed phytoene-containing oleoresin together with one or more pharmaceutically-acceptable, nutraceutically-acceptable or cosmetically-acceptable excipients.

Further details concerning said dosage forms (both oral and topical forms) are provided hereinbelow.

The present invention also encompasses methods for improving or maintaining skin quality and/or protecting the skin form environmental damage (e.g., due to airborne pollutants or UV radiation), wherein said methods comprise the administration of a composition or dosage form, as disclosed above, to a subject in need thereof. In one preferred embodiment, the subject is a human subject. Further details concerning these methods are provided hereinbelow.

### BRIEF DESCRIPTION OF THE DRAWINGS

FIG. 1 presents a flow chart summarizing the process for obtaining a phytoene-rich biomass from a Blakeslea trispora culture

FIG. 2 is a flow chart summarizing the various process steps used to prepare the phytoene-containing oleoresin of the present invention.

FIG. 3 graphically presents results showing the increase in IL-8 production in cultured keratinocytes following treatment with diesel particulate matter, and the inhibition of this effect when the cultured cells are treated with a dilution of the phytoene composition of the present invention.

FIG. 4 is a bar graph showing the inhibitory effect of a phytoene-containing composition of the present invention on reactive oxygen species (ROS) levels in cultured keratinocytes following their exposure to diesel particulate matter.

FIG. 5 is a bar graph showing the inhibitory effect of a phytoene-containing composition of the present invention on IL-8 levels in cultured keratinocytes following their exposure to UVB radiation.

FIG. 6 graphically presents results showing an inhibition of IL-1a production by skin cells in an artificial skin model, when said cells were treated with dilutions of a phytoene-containing composition of the present invention.

FIG. 7 graphically shows the inhibitory effect of phytoene-containing composition dilutions on IL-6 production in an artificial skin model.

FIG. 8 presents data showing the increase in skin brightening caused by the topical application of a composition of the present invention.

FIG. 9 presents data showing the reduction in skin redness caused by the topical application of a composition of the present invention.

FIG. 10 presents data showing the increase in skin firmness caused by the topical application of a composition of the present invention.

FIG. 11 presents data showing the increase in skin elasticity caused by the topical application of a composition of the present invention.

FIG. 12 presents data showing the increase in skin smoothness caused by the topical application of a composition of the present invention.

FIG. 13 presents data showing the improvement in various skin wrinkle parameters following the topical application of a composition of the present invention.

FIG. 14 presents data showing the reduction in transepidermal water loss caused by the topical application of a composition of the present invention.

FIG. 15 presents data showing the reduction in skin irritation following the topical application of a composition of the present invention.

### DETAILED DESCRIPTION OF THE PRESENT INVENTION

As explained hereinabove, in one aspect, the present invention is directed to a solvent-free, non-GMO oleoresin obtained from the mold species Blakeslea trispora, comprising phytoene at a concentration of 10-40% (w/w). In some preferred embodiments, the phytoene concentration of the oleoresin is in the range of 20-30% (w/w). In one preferred embodiment, the phytoene concentration in the oleoresin is about 20% (w/w).

It is to be noted that the term ‘oleoresin’ is generally understood as referring to a semi-solid (or viscous liquid) extract comprising essential and/or fatty oils as well as other, mainly hydrophobic, compounds. Historically, such oleoresins have been obtained exclusively by means of solvent extraction of biological material (e.g., plant material or biomass obtained following fermentation of microorganisms), and the subsequent removal of said solvent. However, in the context of the present invention, which relates to an entirely solvent-free composition, the oleoresin is obtained by means of supercritical fluid extraction, preferably supercritical carbon dioxide extraction. In addition, as mentioned hereinabove, the oleoresin is a “non-GMO” oleoresin, that is, it is not obtained from a genetically-modified organism.

In some embodiments of this aspect of the present invention, the oleoresin further comprises additional biologically-active compounds, notably phytosterols. In some cases, the major phytosterol component of the oleoresin is ergosterol.

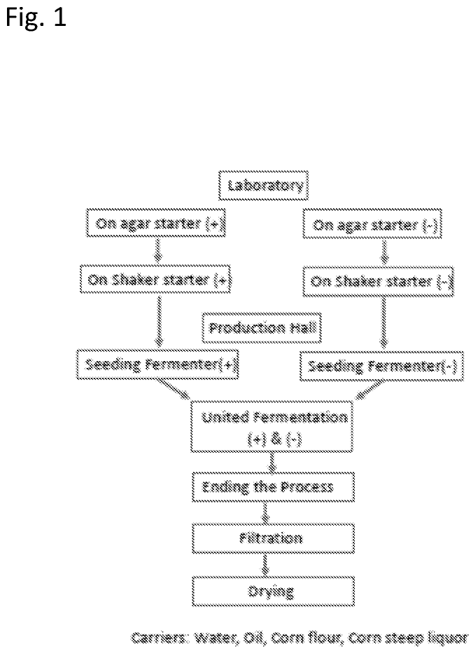

The phytoene-rich biomass is obtained from a B. trispora strain in accordance with the teachings of UA85489 and UA 86556, as explained hereinabove. This process is summarized in the flow chart provided in FIG. 1.

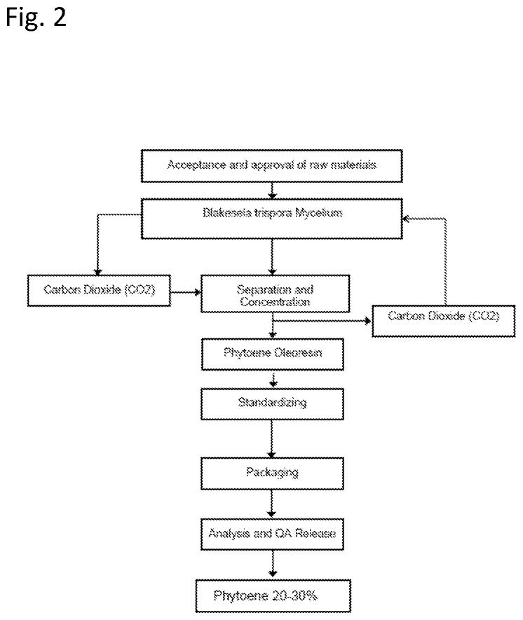

The biomass (usually containing between 5 and 7% (w/w) phytoene and about 40%-50% oil) is then subjected to supercritical carbon dioxide fluid extraction performed at an operating pressure of between 300 and 450 Bar. The extraction was performed at a temperature of about 50° C.-65° C. Following this extraction, an oleoresin (as described hereinabove) containing phytoene at a concentration of generally between 20 and 30% (w/w) and a mixture of phytosterols (of which ergosterol is a major component) at a concentration of about 0.6% (w/w), was obtained. An overview of this process is shown in the flow diagram presented in FIG. 2.

As described hereinabove, the present invention also provides a composition comprising phytoene and phytosterols. In one preferred embodiment, said composition comprises 1% (w/w) phytoene and 0.02% (w/w) phytosterols. In another preferred embodiment, the composition comprises 0.1% (w/w) phytoene and 0.002% phytosterols.

In one embodiment of the above-disclosed methods, the phytoene present in the administered composition comprises 15-cis-phytoene. In some embodiments, more than 50% (w/w) of the phytoene in said composition is 15-cis-phytoene. In other embodiments, more than 90% (w/w) of the phytoene is 15-cis-phytoene.

As mentioned above, the present invention includes within its scope dosage forms comprising the above-disclosed phytoene-containing composition, together with one or more pharmaceutically-acceptable, nutraceutically-acceptable or cosmetically-acceptable excipients. Said dosage forms may be formulated either for topical administration or for oral administration, as will be described in more detail hereinbelow.

In addition, the present invention provides dosage forms comprising a phytoene-containing oleoresin as disclosed and described herein, together with one or more pharmaceutically-acceptable, nutraceutically-acceptable or cosmetically-acceptable excipients. Such dosage forms may be prepared such that they are suitable for topical administration. In one preferred embodiment, however, the dosage form of this type is suitable for oral administration.

In one preferred embodiment, the dosage form is suitable for topical administration (i.e., for application to the skin or mucous membranes of the subject). For example, said composition may be formulated as a cream, lotion, ointment, gel, foam, salve, suspension, oil or solution. Alternatively, or additionally, the composition may be adsorbed into a pad or other delivery device that is suitable for being place on, or adhered to, the skin or mucosal surface.

In some embodiments, the topical dosage form may be formulated as an emulsion, such as an oil-in-water or water-in-oil emulsion. In other cases, the hydrophobic phytoene may be diluted in a biocompatible diluent of natural origin, such as a plant oil (e.g., olive oil, sunflower oil, tomato oil, jojoba oil, and so on), liquid paraffin or other conventional lipophilic diluent (e.g., squalene).

In some preferred embodiments, the concentration of phytoene in the topical dosage form is in the range of 0.01 to 0.5% (w/w). In some preferred embodiments, the phytoene concentration is 0.1%. In other preferred embodiments, the phytoene concentration is 0.02% (w/w).

In another embodiment, the dosage form of the present invention, as disclosed hereinabove, is a dosage form suitable for oral administration. Preferably, such oral dosage forms are selected from the group consisting of tablets, caplets, capsules, lozenges, chewable tablets, suspensions, syrups, oils or solutions.

In some embodiments, an oral composition is in the form of a soft gel capsule. In some embodiments, an oral composition is in the form of a beverage, a shot, a gummy, or a powder. In some embodiments, an oral composition is mixed or assimilated into a food stuff, such as chocolate, ice cream, or others.

Such unit dosage forms comprise a safe and effective amount of the composition. The pharmaceutically-acceptable carriers suitable for the preparation of unit dosage forms for peroral administration are well-known in the art. In some embodiments, tablets typically comprise conventional pharmaceutically-compatible adjuvants as inert diluents, such as calcium carbonate, sodium carbonate, mannitol, lactose and cellulose; hinders such as starch, gelatin and sucrose; disintegrants such as starch, alginic acid and croscarmelose; lubricants such as magnesium stearate, stearic acid and talc. In one embodiment, glidants such as silicon dioxide can be used to improve flow characteristics of the powder-mixture. In one embodiment, coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents. In some embodiments, the selection of carrier components depends on secondary considerations like taste, cost, and shelf stability, which are not critical for the purposes of this invention, and can be readily made by a person skilled in the art.

In one embodiment, the oral dosage form comprises predefined release profile. In one embodiment, the oral dosage form of the present invention comprises an extended-release tablets, capsules, lozenges or chewable tablets. In one embodiment, the oral dosage form of the present invention comprises a slow-release tablets, capsules, lozenges or chewable tablets. In one embodiment, the oral dosage form of the present invention comprises an immediate release tablets, capsules, lozenges or chewable tablets. In one embodiment, the oral dosage form is formulated according to the desired release profile of the pharmaceutical active ingredient as known to one skilled in the art.

As indicated above, in addition to phytoene and other, optional, active ingredients present in the composition of the present invention, the dosage forms provided herein will generally further comprise one or more excipients. These may include, for example, thickeners, wetting agents, stabilizers, colorants, diluents, bulking agents, controlled-release polymers and so on.

The various dosage form types that may be used to formulate the compositions of the present invention are well known to the skilled artisan in this field, and further details are available in standard reference works, such as Remington's Pharmaceutical Sciences, Mack Publishing Co, Easton, Pa, USA, 21^{st} edition (2006).

As explained hereinabove, the present invention also encompasses methods for improving or maintaining skin quality and/or protecting the skin form environmental damage (e.g., due to airborne pollutants or UV radiation), wherein said methods comprise the administration of a composition or dosage form, as disclosed above, to a subject in need thereof.

Thus, in one embodiment, the present invention is directed to a method for protecting the skin of a subject exposed to airborne pollutants, comprising administering a composition comprising phytoene to a subject in need of such protection. Typically, in this embodiment, the composition of the present invention, or a dosage form containing said composition, may be administered topically to the subject's skin, either as a preventive measure, or in order to alleviate or treat existing lesions caused by exposure to diesel particulate matter (DPM) or other airborne contaminants. The composition may be administered either for a short period of time (e.g., for a few days or weeks) in order to prevent the development of new pollutant-related skin lesions, for example, when the subject knows that they will be spending time in an environment in which such pollutants are present. Alternative, the composition may be administered for a longer period of time (e.g., several weeks to several months). In the case that the composition is formulated as a cream, gel, ointment or other relatively viscous dosage form, a small amount (e.g., from about 0.5 to about 10 cm of a cream) is rubbed into the target sites on the subject's skin surface. The frequency of such treatment may be between once a week to three or more times a day, depending on the precise concentration of phytoene and/or other active ingredients in the formulation, the age of the subject, and/or the reasons for administering said formulation (e.g., for prevention of new lesions, prevention of premature skin aging or for treatment of existing lesions).

In other embodiments of this method, the composition of the present method may be administered orally to a subject in need thereof.

It is to be noted that the phrase ‘protecting the skin of subjects exposed to airborne pollutants’ is to be understood as referring to one or more of preventing, alleviating or treating diseases, disorders and premature aging of the skin of the subjects. These diseases and disorders may be any skin condition that is caused or exacerbated by airborne pollutants. In some embodiments, however, said diseases and disorders are selected from the group consisting of acne, atopic dermatitis, urticaria, skin cancers and premature skin aging.

The present invention is also directed to a method for preventing, alleviating or treating skin conditions which are caused by, or related to, exposure to airborne pollutants, wherein said method comprises administering a composition or dosage form of the present invention to a human (or non-human mammalian) subject.

In one embodiment, the above-mentioned skin conditions are inflammatory conditions mediated at least in part by the cytokines IL-1α, IL-8 and/or IL-6.

The term “airborne pollutants” as used in the context of the two above-disclosed methods, is to be understood as including all types of hazardous compounds, substances and agents that may be present in the air, either indoors or outdoors. Said hazardous compounds, substances and agents may be dispersed or dissolved in the air, or present as liquid droplets, aerosols or as solid particulate matter. In the latter case, the particulate matter may have additional liquid or gaseous components adsorbed onto its surface.

In one embodiment of the present invention, the above-disclosed methods are particularly directed to airborne pollutants emitted in the exhaust of motor vehicles, and in particular, such vehicles which are powered by diesel engines. In one embodiment, the above-disclosed methods are particularly directed to the particulate matter present in diesel exhaust (i.e., DPM). In still other embodiments of the presently disclosed methods, the airborne pollutants are emitted from other forms of transportation (e.g., airplanes and diesel train engines) or from industrial plants and factories.

While any suitable phytoene-containing composition may be used to perform the above-disclosed methods of the present invention, in one preferred embodiment, said composition comprises solvent-free, non-GMO phytoene of natural origin.

In another embodiment, the present invention is directed to a method for protecting the skin of a subject exposed to UV radiation, comprising administering a composition comprising phytoene to a subject in need of such protection. The term “UV radiation” refers to all frequencies of UV radiation, particularly those present in sunlight to which the skin of human subjects is exposed, such as radiation in the UVA and UVB ranges.

In a still further embodiment, the present invention is directed to a method for improving one or more aspects of skin quality, comprising administering a composition comprising phytoene to a subject in need of such protection. Many different aspects of skin quality may be improved by this method. However, in one preferred embodiment, said aspects of skin quality are selected from the group consisting of evening and balancing skin tone, reducing wrinkles, enhancing elasticity, protecting natural skin barrier function and improving skin appearance.

In another aspect, the present invention provides a composition as disclosed hereinabove for use as a medicament. In one preferred embodiment, said medicament is for use in protecting the skin of a subject exposed to airborne pollutants. In another embodiment, said medicament is for use in protecting the skin of a subject exposed to UV radiation. In a still further embodiment, said medicament is for use in improving one or more aspects of skin quality, wherein said improvements are preferably selected from the group consisting of evening and balancing skin tone, reducing wrinkles, enhancing elasticity, protecting natural skin barrier function and improving skin appearance.

In some preferred embodiments of the above-disclosed methods and uses, the composition comprising phytoene is a composition as defined hereinabove and claimed hereinbelow. In some embodiments of these methods and uses, the composition comprising phytoene is administered by means of topical application to the skin and/or mucous membranes of the subject. In other embodiments, the composition comprising phytoene is administered the subject by the oral route, preferably formulated as an oral dosage form, for examples as one of the oral dosage forms defined hereinabove.

As disclosed hereinabove, the composition used in the above-disclosed methods comprises phytoene obtained from the mold species. Blakeslea trispora. In one particularly preferred embodiment said B. trispora is as phyt 1+/phyt 1−strain, for example the strain disclosed in UA85489 and deposited at the Depository of the Institute of Microbiology and Virology (named after DK Zabolotny), NAS of Ukraine under deposition number F-100053.

Preferably, the phytoene contained in the compositions used in the above-disclosed methods is obtained from a biomass produced according to the teachings of UA86556. Further details of this process are provided hereinbelow. The teachings of both UA85489 and UA 86556 are incorporated herein in their entirety.

In one preferred embodiment, the composition administered in the above-disclosed methods comprises phytoene at a concentration of between 0.01% (w/w) and 10% (w/w). In a more preferred embodiment, the concentration of phytoene in the composition is in the range of 0.1 to 2.5% (w/w). In one further preferred embodiment, the phytoene concentration in the composition is about 0.1% (w/w). In a yet further preferred embodiment, the phytoene concentration is about 1% (w/w).

In some embodiments of the methods of the present invention, the composition administered further comprises one or more phytosterols. In one embodiment, the said one or more phytosterols includes ergosterol.

The present invention further encompasses the use of a non-GMO, solvent-free phytoene-containing composition as defined herein, in the manufacture of a medicament or cosmeceutical or nutraceutical agent.

In one embodiment, the invention is directed to the use of a non-GMO, solvent-free phytoene-containing composition as defined herein, in the manufacture of a medicament or cosmeceutical or nutraceutical agent for protecting the skin of subjects exposed to airborne pollutants.

In another embodiment, the invention is directed to the use of a non-GMO, solvent-free phytoene-containing composition as defined herein, in the manufacture of a medicament or cosmeceutical or nutraceutical agent for preventing, alleviating or treating skin conditions which are caused by, or related to, exposure to airborne pollutants.

In a further embodiment, the present invention is directed to the use of a non-GMO, solvent-free phytoene-containing composition as defined herein, in the manufacture of a medicament or cosmeceutical or nutraceutical agent for protecting the skin of a subject exposed to UV radiation.

In a still further embodiment, the present invention is directed to the use of a non-GMO, solvent-free phytoene-containing composition as defined herein, in the manufacture of a medicament or cosmeceutical or nutraceutical agent for improving one or more aspects of skin quality. In a preferred embodiment, said improvement is selected from the group consisting of evening and balancing skin tone, reducing wrinkles, enhancing elasticity, protecting natural skin barrier function and improving skin appearance.

All of the various technical features described in connection with the above-disclosed methods apply equally to the different use aspects disclosed herein.

Various features of the presently claimed invention will now be exemplified in the following non-limiting Examples.

### Example 1

### Inhibition of DPM-Induced IL-8 Production in Cultured Cells by a Composition of the Present Invention

### Introduction

The aim of this study was to investigate whether phytoene is capable of inhibiting the DPM-induced increase in the production of the pro-inflammatory cytokine II-8 in cultured keratinocytes. This in vitro system serves as a model for the inflammatory response in skin cells that is observed in subjects that develop skin lesions and accelerated skin aging, following exposure to airborne pollutants.

### Methods

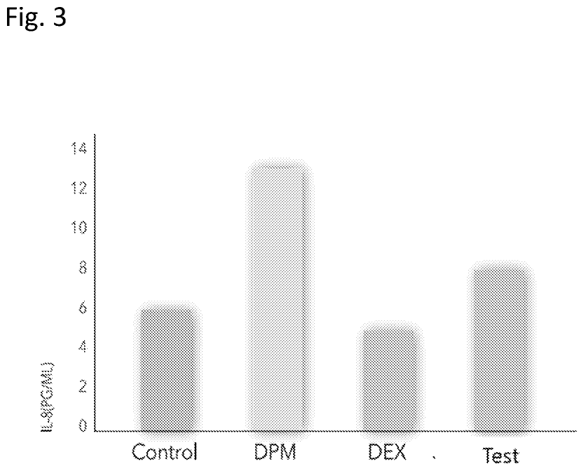

Spontaneously transformed adult human keratinocytes of the HaCaT line were grown and maintained in filtered DMEM supplemented with 100U/ml penicillin and 100 μg/ml streptomycin and 10% FBS. Aliquots of the cells (at a concentration of approximately 2.5×10⁵ cells/ml, were seed in 96-well plates containing 170 μl/well of the aforementioned medium. Each seeded well contained about 42,500 cells. The plates were then incubated at 37° C., with 5% CO₂ for 24 hours. Following this, the medium was aspirated and replaced with a dilution of a 1% (w/w) phytoene-containing composition of the present invention (“Test”) containing a final phytoene concentration of 56 ppm, or the following three controls:
Vehicle control (“Control”, in FIG. 3)
DPM-stimulated control (“DPM”)
Positive control: dexamethasone (“DEX”)

The vehicle for preparing the DPM, DEX and Test solutions was PBS containing 100 μg/ml Diesel Particulate Matter (DPM). All test items and controls were plated in triplicate.

The plates were then incubated for a further 24 hours at 37 C with 5% CO₂. At the end of this incubation period, the media was aspirated and centrifuged at 250×g for 5 minutes, to remove particulate matter. The clear supernatants were then frozen at −70 C until IL-8 analysis could be performed.

Quantitative IL-8 analysis was performed using a commercial ELISA kit (ELISA Max Deluxe set for Human IL-8, supplied by Enco, Catalogue number B287549).

### Results

The results of this study are shown in FIG. 3, which indicates that treatment with DPM alone causes an approximately 2-fold increase in IL-8 production, as compared to the vehicle-only control (negative control). The dexamethasone positive control does not cause a significant change in the basal level IL-8 production of the unstimulated cells. It may be seen, however, that the composition of the present invention (“Test”) caused a marked and statistically significant (p<0.05) inhibition of DPM-stimulated IL-8 production, when compared with the DPM-only group.

These results, obtained using a cultured human keratinocyte model, indicate that phytoene is capable of reducing at least some of the inflammatory response in skin cells that have been exposed to airborne pollutants such as DPM.

### Example 2

### Inhibition of DPM-Induced Reactive Oxygen Species (ROS) Production in Cultured Cells by a Composition of the Present Invention

### Introduction

Airborne particulate components from fossil fuel combustion (such as DPM) can induce oxidative stress via the generation of reactive oxygen species (ROS) that are strongly correlated with airway inflammation and asthma. ROS entities include both free radical and non-free radical oxygen intermediates (peroxides), including superoxide radicals (O₂^{•-}), hydrogen peroxide (H₂O₂), hydroxyl radicals (OH^{•}), and singlet oxygen (102). In this study, the effect of a composition of the present invention on the production of NOS in response to the exposure of cultured keratinocytes to DPM was investigated.

### Methods

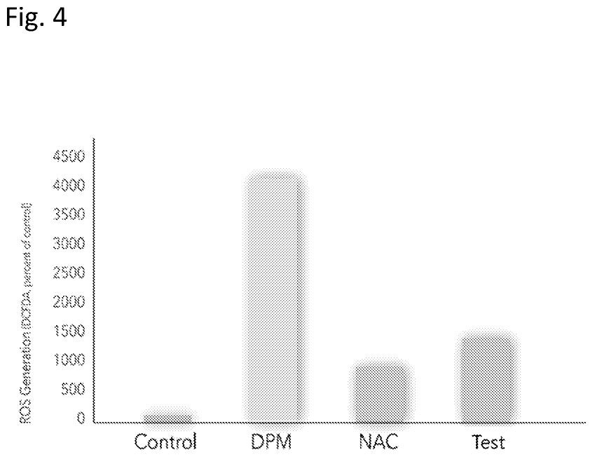

Spontaneously transformed adult human keratinocytes of the HaCaT line were grown, maintained, plated and exposed to control and test substances as described in Example 1, above. The control substances used in this study were:
Vehicle control (“Control” in FIG. 4)
DPM
N-acetyl cysteine (NAC; positive control)

The test substance used was a dilution of the composition of the present invention (i.e., containing non-GMO, solvent free phytoene and jojoba oil), yielding a final phytoene concentration of 56 ppm.

Quantitative analysis of the ROS content of treated and control cell supernatants was performed using a commercially-available kit, which uses a fluorogenic dye (DCFDA) that measures hydroxyl, peroxyl and other intracellular reactive oxygen species ROS activity.

### Results

It will be seen from FIG. 4 that while DPM treatment raised the ROS levels to about 40 times the unstimulated control, the composition of the present invention (“Test”) inhibited this increase in ROS concentration to a highly significant degree and was only slightly less effective than the NAC positive control.

### Example 3

### Inhibition of UVB-Induced IL-8 Production in Cultured Cells by a Composition of the Present Invention

### Introduction

In this study, the effect of a composition of the present invention on the production of IL-8 in UVB-exposed cultured keratinocytes was investigated.

### Methods

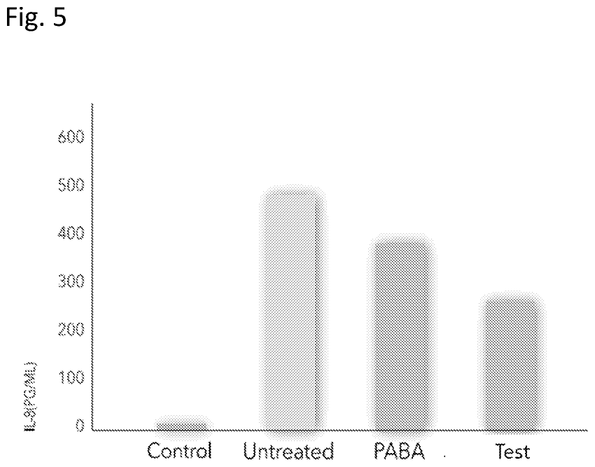

Spontaneously transformed adult human keratinocytes of the HaCaT line were grown, maintained, plated and treated with control and test substances as described in Example 1, above. The cultured cells were irradiated using a UVB lamp in order to generate an inflammatory response in the cultured cells. The control substances used in this study were:
Vehicle control (“Control” in FIG. 5)
UVB exposure only (“Untreated”)
4-Aminobenzoic acid (“PABA”; positive control)

The test substance used was a dilution of the composition of the present invention (i.e., containing non-GMO, solvent free phytoene and jojoba oil), yielding a final phytoene concentration of 56 ppm.

The IL-8 concentration of the culture cell supernatants was measured as described in Example 1, above.

### Results

It will be seen from FIG. 5 that UVB exposure, caused a massive increase in IL-8 production, as compared with vehicle control. It will further be noted that the test composition of the present invention caused a significant reduction in this UVB-stimulated IL-8 production, to a degree greater than the inhibition caused by the positive control (the commercial sunscreen component, PABA).

### Example 4

### Inhibition of DPM-Induced Inflammatory Response in a 3D Reconstructed Skin Model

### Introduction

In order to further investigate the in vitro results presented in Example 1, hereinabove, a reconstructed human epidermis (RHE) model was used as the test platform. The reconstructed skin used in the experiments described in this Example, hereinbelow, which is available commercially under the trade name ‘EpiDerm’, is a highly differentiated 3D tissue model consisting of normal, human-derived epidermal keratinocytes cultured on cell culture inserts. These inserts permit the culture of the reconstructed tissue at an air-liquid interface, thereby readily permitting the evaluation of topically-administered agents on the skin.

### Methods

The EpiDerm reconstructed skin system was obtained from the supplier MatTek (catalogue number EPI-200), and the tissue was handled according to the manufacturer's instructions. Briefly, upon delivery, the tissue was removed from its agar medium, washed and then left to recover overnight at 37 C, in a 5% CO₂ environment.

Then, the tissue was treated with topically-applied DPM (100 μg/ml in PBS, 10 μl/sample) in the presence or absence of the phytoene oleoresin diluted in jojoba wax at concentrations of 0.1, 1 and 2.5%, to a final volume of 30 μl. The following control groups were also set up: naïve tissue (untreated control), vehicle (jojoba wax), DPM-stimulated tissue and N-acetyl cysteine (NAC; positive control). Following addition of the test substances and controls, the reconstructed skin cultures were incubated for a further 48 hours at 37 C, in a 5% CO₂ environment.

At the end of the treatment period, the medium was collected from the tissue control implants and centrifuged at 250×g for 5 minutes, in order to remove particulate matter. The clear supernatants were then frozen at −70 C prior to cytokine analysis.

IL-1a and IL-6 levels in the thawed medium were determined using commercial ELISA kits (ELISA Max Deluxe Set Human IL-1a catalogue number B265424; ELISA Max Deluxe Set Human IL-6 catalogue number B2852558; both supplied by Enco), according to the manufacturer's instructions.

### Results

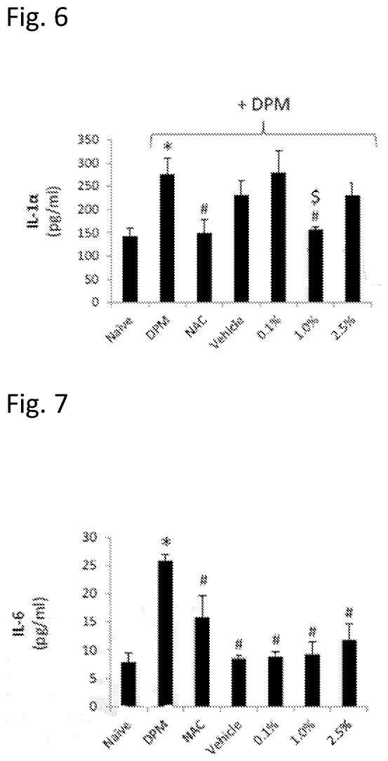

The results of this study are shown in FIGS. 6 and 7. The bar graphs presented in FIG. 6 indicate that in the EpiDerm artificial skin model, some, but not all of the dilutions of the phytoene-containing oleoresin caused a reduction in IL-1a production by the skin cells. It is to be noted that the 1% concentration of this test substance caused a reduction in IL-1a production that was quantitatively comparable to the degree of reduction caused by the anti-inflammatory positive control (N-acetyl cysteine; NAC). Furthermore, the reduction in IL-1a levels caused by this concentration of the test substance was statistically significant (p<0.05) when compared with both the DPM-only and the vehicle-only samples.

FIG. 7 presents the results for IL-6 production. It may be seen from this graph that all concentrations of the phytoene-containing oleoresin dilution that were tested caused a significant reduction in IL-6 production (p<0.05) when compared with the DPM-only samples.

These data demonstrate that exposure of an in vivo skin model to DPM causes an increase in the production of inflammatory mediators, and that treatment with the phytoene-containing composition of the present invention is able to inhibit this aspect of the inflammatory response. These results thus strongly suggest that the composition of the present invention may be of value in preventing, mitigating or treatment skin damage due to the exposure of the skin to environmental pollutants and irritants, such as DPM.

### Example 5

### Clinical Studies of the Effect of a Composition of the Present Invention on Various Aspects of Skin Quality

### Introduction

The following skin quality features were studied in a group of healthy volunteers before and after topical treatment with a composition of the present application:
Skin tone
Skin reddening
Skin firmness
Skin elasticity
Skin smoothness
Wrinkle condition
Skin barrier function
Soothing effect

### Methods and Results

A group of 33 female volunteers (average age 50 years) were randomly selected for this study. The facial skin of the volunteers was treated with a twice daily application of a composition of the present invention (containing 1.5% (w/w) phytoene in jojoba oil) for up to 28 days.

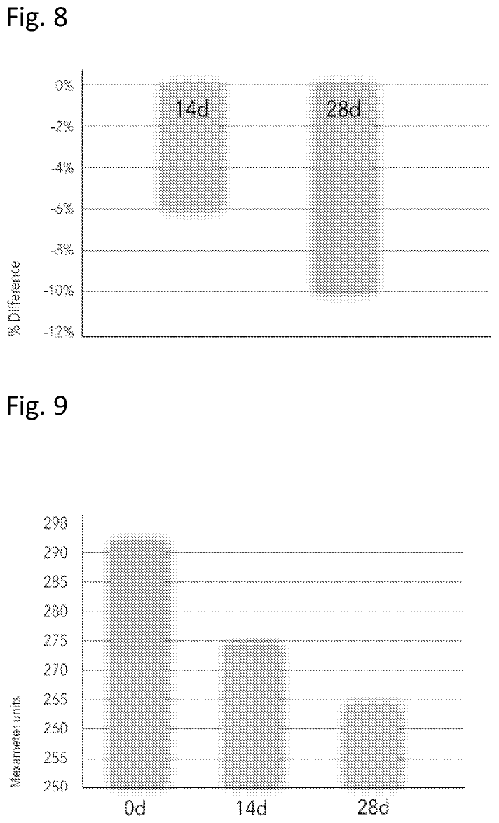

Skin tone was assessed using a Mexameter® MX 18 device (supplied by Courage+Khazaka electronic GmbH, Germany) to evaluate Melanin levels in the subjects. As will be seen from the results shown in FIG. 8, application of the composition of the present invention for 14 days caused a mean 6% reduction in melanic index, while treatment for 28 days caused a mean reduction of 10%, indicating a significant increase in skin brightening as a result of this treatment.

Skin redness was assessed using a Mexameter® MX 18 device. FIG. 9 indicates a significant reduction in erythema following treatment, both at the 14 day and 28-day time points, when compared to the initial measurement prior to treatment.

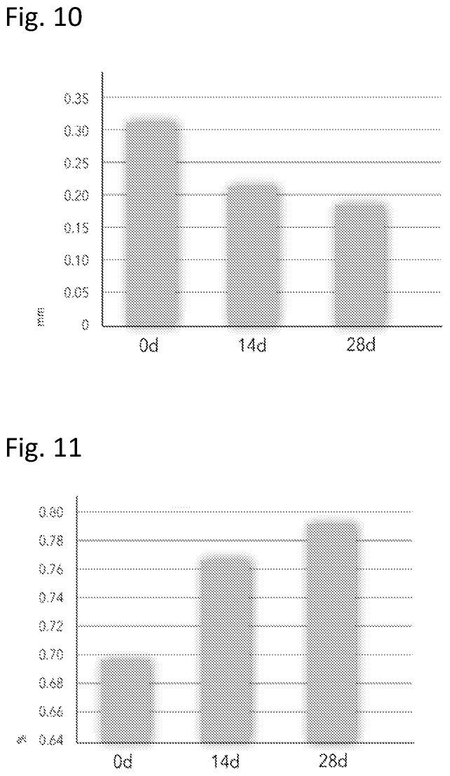

Skin firmness and Skin elasticity were assessed using a suction-based device, the Cutometer® MPA 580 (distributed by Courage+Khazaka electronic GmbH, Germany). FIG. 10 shows an increase in skin firmness (i.e., a decrease in the depth of skin deformability) at both the 14 day and 28-day time points. Similarly, a significant increase in skin elasticity was seen at these two time points, as shown in FIG. 11.

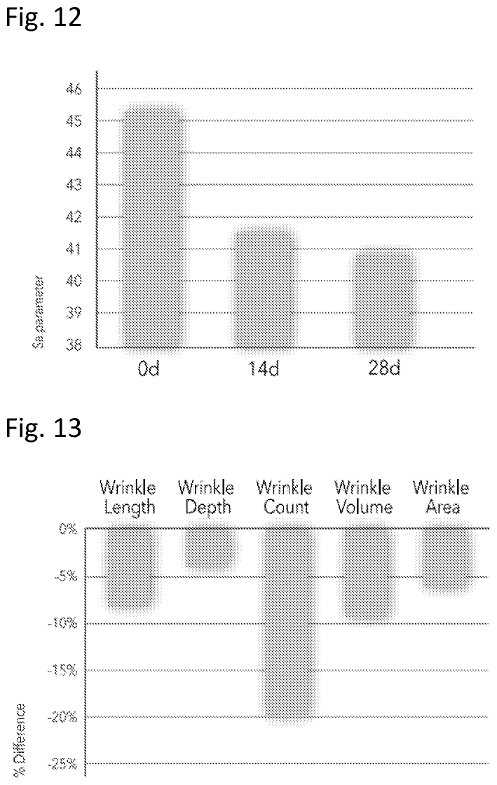

Skin smoothness was assessed using a Primos 3D Lite system (distributed by Canfield Scientific, New Jersey, USA). As shown in FIG. 12, a significant increase in smoothness was seen at both the 14- and 28-day time points.

Wrinkle analysis was performed using a Primos 3D Lite system. FIG. 13 indicates that while all of the various parameters measured (i.e., wrinkle length, depth, count, volume and area) improved after 28 days' treatment, the most significant change (reduction by 20%) was seen with regard to the number of wrinkles.

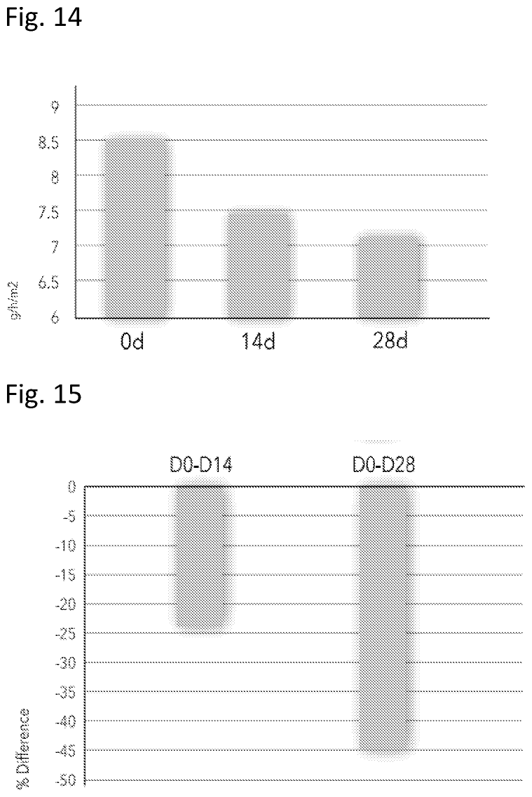

Skin barrier function Skin hydration was used as a measure of skin barrier function and was assessed using a Tewameter® transepidermal water loss (TEWL) device. As shown in FIG. 14, treatment with the composition of the present invention caused a marked decrease in water loss at both 14 days and 28 days.

Skin soothing The ability of the composition of the present invention to soothe irritated skin was assessed using subject-generated scores following application of a 10% lactic acid solution to the skin surface. As seen in FIG. 15, treatment with a composition of the present invention for 14 days resulted in a greater than 20% reduction in irritation. This skin soothing effect was even more marked following 28 days' treatments, with the reported irritation being reduced by 45%.

### Example 6

### Face Cream Formulation Comprising the Composition of the Present Invention

| Trade name | INCI name | % Wt |
|---|---|---|
| D.I. Water | Aqua | 95.6 |
| Lecigel | Sodium Acrylate | 1.8 |
| | Copolymer and Lecithin | |
| Phyto-Jojoba | Simmondsia Chinensis (Jojoba) | 1.5 |
| | Seed Oil C30-45 Olefin | |
| Tween 20 | Polysorbate 20 | 0.1 |
| Euxyl PE | Phenoxyethanol (and) | |
| 9010 | Ethylhexylglycerin | 1 |
| Total | | 100 |

A phytoene-containing oleoresin of the present invention, prepared as described hereinabove and summarized in FIG. 1, is mixed with the face cream formulation prepared from the ingredients summarized in the above table. The final phytoene concentration of the formulation is 0.1% (w/w).

### Example 7

### Hair Cream Formulation Comprising the Composition of the Present Invention

| Trade name | INCI name | % Wt |
|---|---|---|
| D.I. Water | Aqua | 88.3 |
| Palmerol 1899 | Stearyl alcohol | 1.4 |
| Silicone 350 | Dimethicone | 5 |
| Phyto-Jojoba | Simmondsia Chinensis (Jojoba) | 1.5 |
| | Seed Oil C30-45 Olefin | |
| Citric Acid | Citric Acid | 0.1 |
| Euxyl PE 9010 | Phenoxyethanol (and) | 0.7 |
| | Ethylhexylglycerin | |
| Salcare SC 96/ | Polyquaternium-37 (and) | 2 |
| Cosmedia | Propylene Glycol | |
| CTH (E) | Dicaprylate/Dicaprate (and) | |
| | PPG-1 Trideceth-6 | |
| Keravis PE-LQ-(WD) | Aqua (and) Hydrolyzed | 1 |
| | Vegetable Protein PG- | |
| Total | Propyl Silanetriol | 100 |

A phytoene-containing oleoresin of the present invention, prepared as described hereinabove and summarized in FIG. 1, is mixed with the hair cream formulation prepared from the ingredients summarized in the above table. The final phytoene concentration of the formulation is 0.1% (w/w).

### Example 8

### Orally-Administrable Soft-Gel Capsule Containing Phytoene Composition of the Present Invention

Phytoene oleoresin soft gel capsules are prepared according to standard procedures known to the skilled artisan. Each capsule contains an oleoresin prepared according to the present invention, such that the final concentration of phytoene in said capsule is in the range of 0.01-10% (w/w).

### Example 9

Effect of Phytoene oleoresin Composition in Protection of Healthy Skin from UV-Induced Photo-Oxidative Damage

### Methodology

A double blind, placebo controlled parallel group clinical biostudy. After a 5-week run-in period, during which the intake of phytoene-rich foods and antioxidant supplements is restricted, subjects are randomized to receive formulation 1 soft gel capsules or identically appearing soft gel capsules as placebo, daily for 12 weeks.

### Number of Subjects (Planned and Analyzed)

60 subjects to complete the study and be analyzed.

### Diagnosis and Main Criteria for Inclusion

Healthy males and females, 20 to 65 years of age, with Fitzpatrick skin type I to IV, BMI≤30 kg/m² and healthy eating habits.

### Test Product, Dose and Mode of Administration

1 soft gel capsule (as described in Example 8, above) administered orally daily.

### Reference Therapy, Dose and Mode of Administration

Placebo administered orally daily.

### Duration of Treatment

12 weeks

### Protection Against Erythema Measured by Chromometry

Chromometry is a method of measuring skin color using a chromometer. The color of the skin is measured using a L*a*b* scale, where L* reflects the luminance of a color on a gray scale, a* reflects redness versus greenness, and b* reflects blueness versus yellowness.

The subjects are tested for the protection from UV irradiation induced erythema. Each subject is UV irradiated on an area of his skin and the development of erythema is measured 24 hours after the irradiation.

For this analysis, Δa* was defined as the difference between erythema development levels at 24 hours after UV irradiation before supplementation and erythema development levels at 24 hours after UV irradiation after supplementation. A statistically significant change in erythema formation is expected between the formulation 1 and placebo groups.

### Cytokine Levels

Mean IL-1alpha, IL-6, IL-10 and TNF-alpha levels are assessed by polymerase chain reaction (PCR). The subjects participating in the clinical study are assayed for the levels of IL-1alpha, IL-6, IL-10 and TNF-alpha. Samples are obtained after UVB irradiation prior to randomization and after UVB irradiation done after 12 weeks of treatment with formulation 1 or placebo.

Expected results: Analysis of cytokine mRNAs from skin biopsies is statistically significant different between treatment groups.

### Example 10

A Composition of the Present Invention Reduces Secretion of Degradative Enzymes from Stimulated Neutrophils

In order to study the effect of a composition of the present invention on the release of enzymes that have a potential to cause damage to collagen, the inventors first determine the kinetics of the release of such enzymes from activated neutrophils. Neutrophils are activated by 1.00 ng/ml TNFa or 1.00 ng/ml 11.8 that are released from the skin cells during exposure to UV and activated. In addition, neutrophils are activated with 5×10⁷fMLP for comparison.

Neutrophils are to be activated by TNFa or 11.8 for 4 hr and by fMLP for 30 min, in order to study the effect of the phytoene-containing composition of the present invention on the release of MMP-9 or MPO. The composition is added to the neutrophils for 10 min at 37° C. before activation. Inhibition of the release of cytokine from stimulated neutrophils is measured and a dose dependent inhibition is expected

### Example 11

### The Phytoene-Containing Composition of the Present Reduces Collagen Loss in Fibroblast-Neutrophils Co-Cultures

In order to study the effect of a composition of the present invention on the damage of collagen-3 induced by activated neutrophils the inventors use optimal conditions of cultured fibroblasts and of neutrophils. 1×10⁵ fibroblasts are plated for 24 hr to obtain confluent cultures. Cell viability is measured in each treatment.

Neutrophils are incubated with a composition of the present invention for 10 min at 37° C. before being added to the cultures for 24 hr. Quantification of collagen-3 levels in the cultures is to be determined by densitometry.

It is expected that the presence of the composition of the present invention in the co-cultures will have a dose-dependent increase in the secreted pro-collagen-3.

Treatment of NHDF cells with 50 mM H₂O₂ results in about a 10-fold reduction in collagen lal secretion. It is expected that the composition of the present invention will completely abolish the reduction in collagen secretion, suggesting a role for the composition in increasing skin collagen levels and reducing skin ageing, in vivo.

1. A solvent-free, non-GMO oleoresin obtained from Blakeslea trispora, comprising phytoene at a concentration of 10-40% (w/w).

2. The oleoresin according to claim 1, wherein the phytoene concentration is 20-40% (w/w).

3. The oleoresin according to claim 1, wherein the phytoene concentration is 20-30% (w/w).

4. The oleoresin according to claim 1, wherein said oleoresin further comprises one or more phytosterols.

5. The oleoresin according to claim 4, wherein the one or more phytosterols comprise ergosterol.

6. The oleoresin according to claim 5, wherein the concentration of ergosterol is in the range of 0.5 to 1% (w/w).

7. The oleoresin according to claim 6, wherein the concentration of ergosterol is about 0.6% (w/w).

8. A composition comprising an oleoresin according to claim 1 and Simmondsia Chinensis (Jojoba) seed oil.

9. The composition according to claim 8, wherein the concentration of phytoene is in the range of 0.01% to 10% (w/w).

10. The composition according to claim 9, wherein the concentration of phytoene is in the range of 1 to 2% (w/w).

11. A dosage form comprising a composition according to claim 8, and one or more pharmaceutically-acceptable, nutraceutically-acceptable or cosmetically-acceptable excipients.

12. The dosage form according to claim 11, wherein said dosage form is suitable for topical administration.

13. The dosage form according to claim 12, wherein said dosage form is selected from the group consisting of cream, lotion, ointment, gel, foam, salve, suspension, oil or solution.

14. The dosage form according to claim 12, wherein the concentration of phytoene in said dosage form is in the range of 0.01 to 0.5% (w/w).

15. The dosage form according to claim 11, wherein said dosage form is suitable for oral administration.

16. The dosage form according to claim 15, wherein said dosage form is selected from the group consisting of tablets, caplets, capsules, syrups, oils, soft gel capsules, lozenges, chewable tablets, suspensions or solution.

17. A method for protecting the skin of a subject exposed to airborne pollutants, comprising administering a composition comprising phytoene to a subject in need of such protection.

18. A method for protecting the skin of a subject exposed to UV radiation, comprising administering a composition comprising phytoene to a subject in need of such protection.

19. A method for improving one or more aspects of skin quality, comprising administering a composition comprising phytoene to a subject in need of such protection.

20. The method according to claim 19, wherein the improvement in one or more aspects of skin quality are selected from the group consisting of evening and balancing skin tone, reducing wrinkles, enhancing elasticity, protecting natural skin barrier function and improving skin appearance.

21. The method according to claim 17, wherein the composition comprising phytoene is a composition.

22. The method according to claim 17, wherein the composition comprising phytoene is administered to the subject by means of topical application.

23. The method according to claim 22, wherein the composition is formulated as a topical dosage form.

24. The method according to claim 17, wherein the composition comprising phytoene is administered orally to the subject.

25. The method according to claim 24, wherein the composition is formulated as an oral dosage.

26. A solvent-free, non-GMO oleoresin obtained from Blakeslea trispora, comprising phytoene at a concentration of 20-30% (w/w), obtained by means of a process comprising the steps of:
a) Cultivating a phyt 1+/phyt 1−strain of the mold species Blakeslea trispora;
b) Obtaining a biomass from said cultivated B. trispora;
c) Subjecting said biomass to supercritical carbon dioxide fluid extraction performed at an operating pressure of between 300 and 450Bar, and an operating temperature of about 50° C.-65° C., thereby yielding an oleoresin comprising phytoene at a concentration of 20 to 30% (w/w) and ergosterol.

27. The oleoresin obtained according to claim 26, wherein the concentration of ergosterol is about 0.6% (w/w).

28. A dosage form comprising an oleoresin according to claim 1, and one or more pharmaceutically-acceptable, nutraceutically-acceptable or cosmetically-acceptable excipients.

29. The dosage form of claim 28, wherein said dosage form is suitable for oral administration.
